(19) United States
(12) Patent Application Publication
(10) Pub. No.: US 2011/0270083 A1
(43) Pub. Date: Nov. 3, 2011

(54) SYSTEM AND METHOD FOR DYNAMIC METAL DISTORTION COMPENSATION FOR ELECTROMAGNETIC TRACKING SYSTEMS

(75) Inventor: Eric Shen, Croton-on-Hudson, NY (US)

(73) Assignee: KONINKLIJKE PHILIPS ELECTRONICS N.V., EINDHOVEN (NL)

(21) Appl. No.: 13/142,860

(22) PCT Filed: Nov. 10, 2009
(86) PCT No.: PCT/IB09/54997

§ 371 Date: Jun. 30, 2011

Related U.S. Application Data
(60) Provisional application No. 61/141,404, filed on Dec. 30, 2008.

(51) Int. Cl. A61B 5/05

(52) U.S. Cl. 600/424

(57) ABSTRACT

A method and system for dynamic metal distortion compensation using an Electromagnetic Tracking System (EMTS) (10) using an electromagnetic field from an electromagnetic field generator (12). A plurality of fiducial markers (14) are provided, each having at least one electromagnetic sensor (26), the electromagnetic sensors oriented in a plurality of sensor orientations, and at least some of the sensors being located proximal to a volume of interest. The fiducial markers (14) are imaged to provide their position in image space. Position readings of the electromagnetic sensors (26) are monitored using the EMTS. A metal distortion correction function is calculated by comparing the positions of the fiducial markers in image space to the positions of the electromagnetic sensors. A medical device (16) moving through the volume of interest is also tracked using the EMTS, and the distortion correction function is applied to medical device position readings to compensate for the distortion.

The present application relates to systems and methods for dynamic metal distortion compensation for electromagnetic tracking systems, particularly using active fiducial markers.

The outcomes of minimally invasive medical procedures can be improved by the use of electromagnetic tracking systems (EMTS) to track the location of medical instruments and display this information on medical images, thereby helping to guide the medical instrument to a target location in the anatomy.

An EMTS works by using an electromagnetic (EM) field generator, which creates a local EM field at the site of the procedure, and a medical instrument containing a suitable miniaturized sensor coil. A current is induced in the sensor coil that is a function of the position and orientation of the sensor coil relative to the EM field generator. The EMTS can compute the position of the sensor coil, and therefore the position of the medical instrument. A particular advantage of EMTS is that line of sight is not required, because the EM field can penetrate the human body mostly undisturbed. Therefore EMTS is especially suitable for tracking needles or catheters inside the anatomy.

One of the main problems with using EMTS in a medical environment is the presence of metallic conductive or ferromagnetic objects in proximity to the EM field. These objects create distortions, or metal artifacts, which create errors in the position and orientation tracking of the medical instrument. In a medical environment, there are many objects that contribute to metal artifacts in the EMTS. The main sources of distortion come from the medical imaging equipment upon which the patient lies (e.g. CT gantry, CT table, X-ray C-arm, etc.). Another source of distortions is moveable medical equipment or tools (ECG monitors, metallic tools, etc.) that come within the vicinity of the EMTS. These sources distort the EM field and thus distort the position and orientation readings from the EMTS, introducing tracking errors. Such errors may directly affect the outcome of a medical procedure using the EMTS. Currently, the clinical utility of EMTS is limited because the positional and orientational accuracy of the EMTS cannot be guaranteed in the presence of metal distortions.

US 2005/0107687 to Anderson proposes a system and method for distortion analysis and reduction in an EMTS. A tracking modification unit relies on a predetermined distortion model for each specific tool or instrument being tracked. The predetermined model is developed through an analysis process including field mapping and/or modeling/simulation, which also takes into account sensor placement and shielding. The tracking analysis unit generates a map and/or a model of a distortion characteristic of the instrument, which is essentially a lookup table for the instrument. This system does not attempt to reduce distortions created by static objects within the environment.

US 2008/0079421 to Jensen proposes a static mapping of distortion fields created by objects within an environment. An array of EM sensors is positioned within the volume of interest and the array of sensors is sampled to acquire signals representative of the location of the EM sensors within the array. As the array includes a fixed, known geometry, the EM field distortion can be calculated. This system cannot be used in real time during a medical procedure, nor can it take into account field distortions created by moving medical instruments and tools within the volume of interest.

WO 2007/113719 to Shen et al. proposes a system for local metal distortion correction for improving the accuracy of EMTS in a medical environment. The system contains an electromagnetic field generator monitoring a medical device having a suitable sensor coil wherein a correction function, derived from an error correction tool, is applied to the position and orientation readings of the sensor coil. The error correction tool consists of a number of electromagnetic sensors arranged in a fixed and known geometric configuration and is placed surrounding the site of the medical procedure. Sensor data is displayed on an imaging system. In addition, a distortion mapping can be undertaken utilizing optical sensors for relative positioning readings along with an electromagnetic tracking system sensor.

A need remains for systems and methods that can effectively compensate for metal distortions in real time, to improve the accuracy and reliability of EMTS in a medical environment. It is therefore desirable to provide systems and methods that can compensate both for static distortions created by the environment, and for distortions created by the medical instruments and tools themselves.

The Summary is provided to comply with U.S. rule 37 C.F.R. §1.73, requiring a summary of the invention briefly indicating the nature and substance of the invention. It is submitted with the understanding that it will not be used to interpret or limit the scope or meaning of the claims.

In accordance with one aspect of the exemplary embodiments, a method for dynamic metal distortion compensation using an Electromagnetic Tracking System (EMTS) includes generating an electromagnetic field from at least one electromagnetic field generator. A plurality of fiducial markers are provided, each fiducial marker comprising at least one electromagnetic sensor, the electromagnetic sensors oriented in a plurality of sensor orientations, and at least some of the sensors being located proximal to a volume of interest. The fiducial markers are imaged to provide at least a baseline position of the fiducial markers in image space. The method further includes monitoring position readings of the plurality of electromagnetic sensors using the EMTS, and calculating a metal distortion correction function by comparing the positions of the fiducial markers in image space to the position readings of the electromagnetic sensors in the electromagnetic field. Position readings of a medical device moving through the volume of interest are monitored using the EMTS, the device having at least one electromagnetic sensor. The distortion correction function is then applied to the medical device position readings to compensate for said metal distortion. The correction is dynamic and in real-time, which allows for compensations for objects/distortions brought into the vicinity during the procedure.

The positioning of at least one fiducial marker is alterable during the position monitoring. At least some of the fiducial markers are placed on a frame surrounding at least a portion of a patient's body during a medical procedure, and/or at least some of the fiducial markers are placed directly onto a patient's skin during a medical procedure. Alternatively, or in addition, at least one of the fiducial markers may be placed internally in a patient's body during a medical procedure.

Some of the position readings of the plurality of electromagnetic sensors are selected to contribute to the metal distortion correction function. The selection of the electromagnetic sensors can be dynamically based on selection criteria. The selection criteria can include selecting sensors with orientations closest to the orientation of the tracked medical device to calculate the compensation. In other arrangements, the selection criteria can include selecting sensors with spatial locations proximal to the spatial location of the tracked medical device to calculate the compensation. In further arrangements, the selection criteria can include selecting sensors with spatial locations proximal to a target location within a patient's body to calculate the compensation. In yet further arrangements, the selection criteria can include selecting sensors based on the geometry of the relative spatial locations of the tracked medical device and a target location within a patient's body to calculate the compensation. In all arrangements, the selection criteria can change as at least one of the orientation and spatial location of the tracked medical device changes.

The method for calculating the metal distortion correction function can be selected based upon the selection of electromagnetic sensors. For example, a global transformation (affine) calculation method can be used, an interpolation calculation can be used, a global transformation calculation method can be used if the tracked medical device lies outside a geometric coverage of the selected sensors, and/or an extrapolation calculation method is used if the tracked medical device lies outside a geometric coverage of the selected sensors. The method for calculating the metal distortion correction function can be dynamically changed as the selection of electromagnetic sensors is changed due to movement of the tracked medical device.

In accordance with another aspect of the exemplary embodiments, a system for dynamic metal distortion compensation using an Electromagnetic Tracking System (EMTS) includes at least one electromagnetic field generator for generating an electromagnetic field. A plurality of fiducial markers, each fiducial marker comprising at least one electromagnetic sensor, the electromagnetic sensors oriented in a plurality of sensor orientations, and at least some of the sensors being located proximal to a volume of interest, the fiducial markers being visible in image space. A processor is included for calculating a metal distortion correction function by comparing positions of the fiducial markers in image space to position readings of the electromagnetic sensors in the electromagnetic field. At least one electromagnetic sensor is attached to a medical device. The processor applies the calculated distortion correction function to said medical device position readings to compensate for the metal distortion.

At least some of the fiducial markers are provided on a frame adapted to surround at least a portion of a patient's body during a medical procedure. At least some of the fiducial markers comprise a plurality of electromagnetic sensors, and the plurality of electromagnetic sensors in such fiducial markers can have differing sensor orientations.

The above-described and other features and advantages of the present disclosure will be appreciated and understood by those skilled in the art from the following detailed description, drawings, and appended claims.

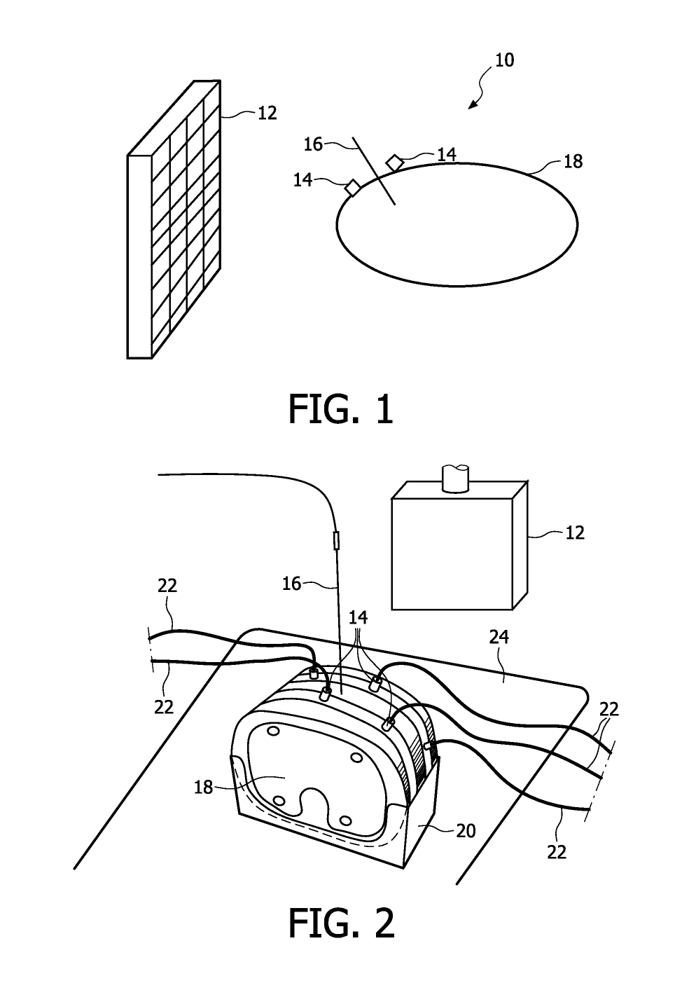

FIG. 1 is a general arrangement of components of the invention.

FIG. 2 shows an abdominal phantom with active fiducial markers attached to a frame.

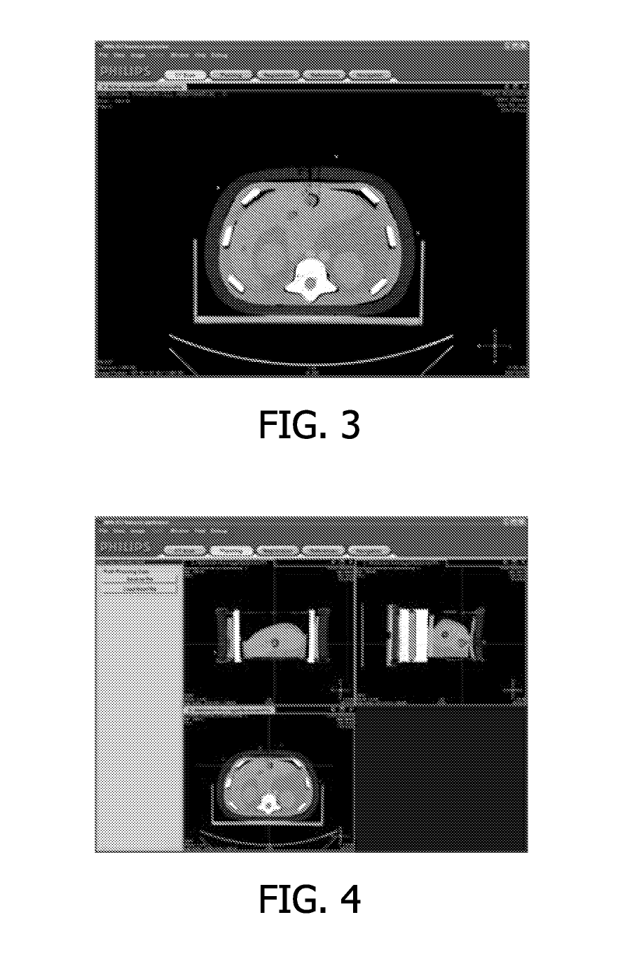

FIG. 3 shows image acquisition

FIG. 4 shows path planning.

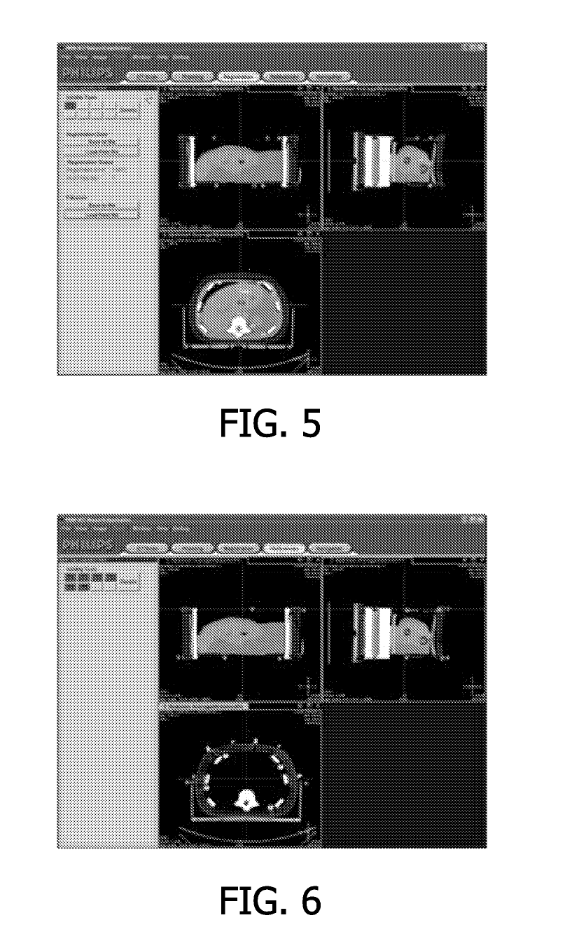

FIG. 5 shows baseline registration.

FIG. 6 shows the identification of active fiducial markers in image space.

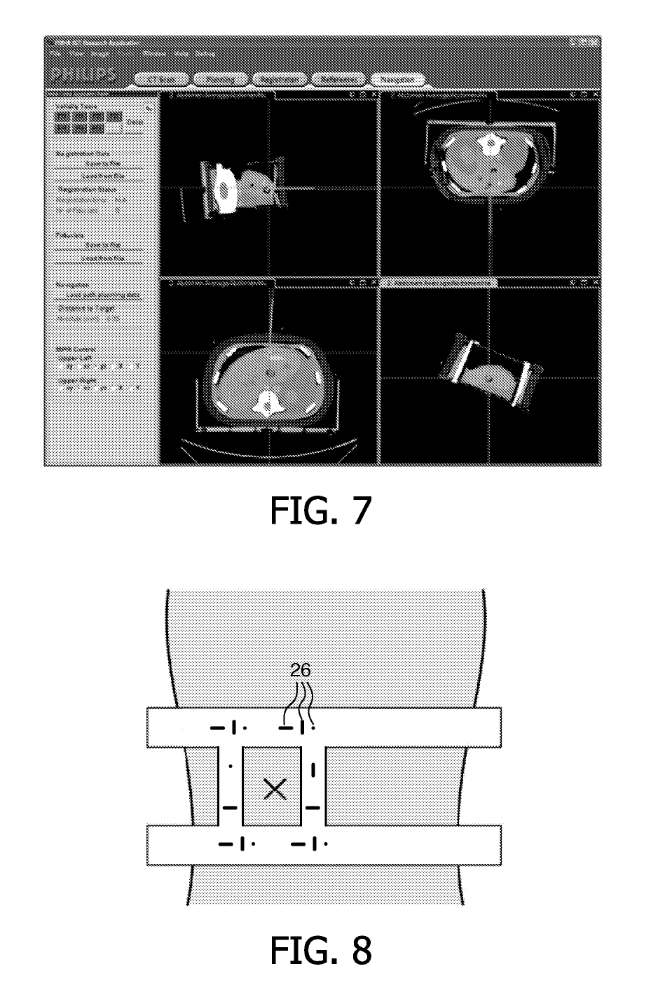

FIG. 7 illustrates navigation with EM distortion compensation.

FIG. 8 illustrates an example showing active fiducials in three different orientations, arranged around a target.

The present disclosure relates to electromagnetic tracking systems (EMTS) for medical devices and other structures. It should be understood by one of ordinary skill in the art that the exemplary embodiments of the present disclosure can be applied to many types of structures, including, but not limited to use in catheter tracking in cardiac and vascular application, oncology applications such as needle biopsies, radio-frequency ablations, cryoablations, prostate cancer therapies, and the like.

Referring initially to FIG. 1, an electromagnetic tracking system (EMTS) 10 having an electromagnetic (EM) field generator 12 is illustrated. In one arrangement, the generator 12 can create a local EM field capable of tracking sensor data from EM sensors contained within active fiducial markers 14 and a medical instrument 16. The markers 14 are arrayed around the patient's body 18. The markers 14 are visible in a medical image space, and also contain a sensor coil to provide position and orientation information in the EM tracking space, such that they are also locatable within the EM tracking space. During a medical procedure, the instrument 16 typically penetrates a patient's body 18 beneath the skin to a target location. An EM sensor coil is embedded in the instrument 16, for example, close to the tip if the instrument 16 is or includes a needle. A current is induced in the sensor coil that is a function of the position and orientation of the sensor coil relative to the EM field generator 12. The EMTS 10 can compute the position of the sensor coil, and therefore the position of the medical instrument 16. A particular advantage of EMTS is that line of sight is not required. Therefore EMTS is especially suitable for tracking needles or catheters inside the anatomy.

Referring now to FIGS. 2-8, during a medical procedure, the active fiducial markers 14 can be placed on the surface of the patient's skin, or on a fixed frame 20 designed to go around the patient 18 (in the illustrated arrangement, an abdominal phantom 18 is shown in place of a patient, as may be used for testing purposes). The markers 14 can be placed to encompass a suitable area in the vicinity of the entry point on the patient's skin or in the vicinity of the target location within the patient's body. The markers may be connected to the EMTS 10 by wires 22. The patient 18 and frame 20 can be positioned on a table 24, with the EM generator 12 positioned above the table 24, or at any suitable location. Images are acquired of the relevant patient anatomy. The active fiducial markers 14 are clearly identifiable in the medical images and the positions of the markers in the image space are determined (via software application). This forms a baseline truth for the positions of the active fiducial markers 14, and position readings for the markers 14 are thus acquired by the EMTS. These positions from the EMTS are used to calculate the compensation by comparing the EMTS positions to the baseline truth image positions. If there is a source of metal distortion, the position of one or more of the active fiducial markers 14 will be distorted, or incorrect. Comparisons with the baseline image positions allows a correction to be calculated. The correction can be implemented in a variety of ways including rigid registration, affine registration, and numerous interpolation methods.

It has been found that fiducial markers 14 with a single sensor orientation such as tangential to the skin result in relatively poor registrations, mainly because ultimately the tracked medical instrument such as a needle is inserted normal to the skin surface, and thus the sensor is normal to the surface of the skin. When markers are fixed to the patient's skin with the sensor inside oriented normal to the skin surface, the active fiducial markers 14 can be used to achieve a useable correction or compensation, to a first order. The effectiveness is optimal when the tracked medical instrument 16 is oriented in the same direction as the sensors in the active fiducial markers. However, it is not always or even often the case that the tracked medical device and the sensors in the active fiducial markers 14 can be closely aligned.

In a first arrangement, the disclosed system and method uses multiple arrays of active fiducial markers 14, each array having a different sensor orientation. In another arrangement, the disclosed system and method can use a plurality of active fiducial markers 14 having varying orientations, which are not necessarily organized into arrays of a specific orientation. A selection is made of either the array of active fiducial markers 14 with the closest sensor orientation to the tracked medical instrument 16, or of the individual active fiducial markers 14 with the closest sensor orientation to the tracked medical instrument 16. The selected array or sensors are then used to calculate the distortion correction. The active fiducial markers 14 can be placed around the patient's body without precision, that is, a priori location is not needed, because the markers 14 only need to be identifiable in the medical image for a baseline position used to calculate the compensations. This gives the freedom to reposition the markers if necessary during the medical procedure.

By placing the active fiducial markers 14 on a frame 20 that surrounds the patient 18, the effects of respiratory motion can be eliminated. If the active fiducial markers 14 are in constant motion due to respiration, this affects the ability to calculate compensations. If respiratory motion can be estimated, then the active fiducial markers 14 may be placed directly on the patient's skin. Position readings from the active fiducial markers 14 would have to coincide with the inspiration level during the acquisition of the image. This can be accomplished through a gating procedure if the patient is on a respirator, or a bellows device could be used. Alternatively, an internal active fiducial marker, or similar marker could be used to estimate the respiratory state using the EMTS or similar tracking system.

To use the EMTS, the patient is first imaged (see FIG. 3) using any suitable imaging system. The target path for the interventional medical procedure is then identified (see FIG. 4). These two steps may be carried out immediately prior to the start of the medical procedure, during the procedure, or may be carried out in advance of the procedure. A baseline registration between the image space and the EM tracking space can be obtained (see FIG. 5). The baseline registration is an initial registration between the image space and the EM tracking space. This step is optional because the transformation between the two spaces can be calculated using the active fiducial markers 14 during the compensation calculation. However, performing this step provides a baseline transformation between the image and EM tracking spaces in the event that the calculation of the EM compensation fails. Furthermore, this step is useful if the software application enables user selection of EM distortion compensation (i.e. to turn it on or off).

The locations of the active fiducial markers 14 are then identified in the image space (see FIG. 6). The EMTS position information of the active fiducial markers 14 is then read, and the EMTS calculates the transformation or interpolation between the image space positions and the EMTS position, which implements the distortion compensation. Once the distortion compensation has been calculated, the image can be corrected, and corrected images can be provided to the physician so that interventional navigation can be carried out with real-time distortion compensation (see FIG. 7). Orientation of the tracked medical instrument 16 is thus monitored in real-time.

The compensation is calculated using position readings from sensors in the active fiducial markers 14 with the same (within a threshold) orientation as the tracked medical instrument 16. In most cases, the orientation of the tracked medical instrument 16 is not fixed, because it changes dynamically as the medical instrument 16 is repositioned. Thus, the use of a plurality of sensors with different orientations allows sensors with orientations closest to the orientation of the tracked medical instrument 16 to be selected to calculate the compensation. Alternatively, the sensors with the closest proximity to the medical instrument 16 can be selected, or the selection may be based on the geometric position of the sensors to the medical instrument 16. As the orientation of the tracked medical instrument(s) 16 changes, the appropriate sensors can be selected dynamically to calculate the compensation. In all cases, a minimum number of sensors must be used to calculate the correction, although the actual minimum number will depend on the method used to perform the calculation.

The selection of the sensors that are used for the calculation may be used to determine the compensation method employed for the correction. For example, if only a few sensors meet the selection criteria, the compensation might be implemented by a global affine transformation. However, if a sufficient number of sensors are selected with the appropriate geometrical coverage, an interpolation approach may be used. In some arrangements, when the orientation of the tracked medical device does not correspond exactly to the orientation of a minimum number of active fiducial sensors, an interpolation can be calculated from the sensors most closely matching in orientation. Similarly if the tracked medical instrument 16 falls outside the geometrical coverage of the available sensors, a global transformation approach might be better than an extrapolation approach.

The speed or frequency of the compensation is only limited by two events, acquiring the position readings from the active fiducial markers 14, and the calculation of the compensation. Depending on the number of active fiducial markers used, the speed of the EMTS, and the compensation algorithm used, one compensation can be done in fractions of a second. True continuous real-time compensation may or may not be necessary in a clinical environment.

One approach to sensor selection for use in the compensation calculation is to use a plurality of arrays of sensors where each array contains a number of sensors of substantially the same orientation, or which are closest in proximity to the medical instrument 16. Based on the orientation of the tracked medical instrument 16, the appropriate array of sensors is selected to calculate the compensation. The selection can be done via software identification of the appropriate array, or by a hardware multiplexer/selector. Each individual array of sensors has its sensors spaced appropriately around the target site. A second approach is to use a number of individual sensors (not grouped into arrays) with different orientations. Based on the orientation of the tracked medical instrument 16, the sensors having orientations closest to the orientation of the tracked medical instrument 16 are selected to calculate the compensation. Selection would most likely be done via software identification of the appropriate sensors.

An example simplified illustration of a sensor arrangement is given in FIG. 8 with groups of sensors 26 in three different orientations (the sensors are represented by lines indicative of their orientation). Each group of sensors 26 can be located within one active fiducial marker 14, or each active fiducial marker 14 can contain one sensor, and be grouped together in an array. The number of orientations need not be restricted to three, for example two could be used, or many more than three.

In another arrangement, at least one fiducial marker 14 can be temporarily placed internally in the patient during the procedure, for example, close to the medical instrument 16 that is being tracked, close to the target, or in any suitable position to improve the accuracy of the compensation.

Electromagnetic tracking is a means of improving medical procedures including catheter tracking in cardiac and vascular applications, oncologic applications such as needle biopsies, radio-frequency ablations, cryoablations, prostate cancer therapies, etc. The errors induced by metal interference can affect the accuracy of medical procedures using electromagnetic tracking systems. By providing real-time dynamic error compensation, the disclosed method and system improve the accuracy of EM tracked medical procedures, and makes the use of EMTS more realistic and practical, in turn creating many opportunities for integrating medical imaging with medical device tracking in minimally invasive applications. These medical applications include the use of CT systems, X-ray systems, ultrasound systems—and the technology is generically applicable to almost any situation where a physician needs to guide a medical device to a location within the anatomy.

The invention, including the steps of the methodologies described above, can be realized in hardware, software, or a combination of hardware and software. The invention can be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The invention, including the steps of the methodologies described above, can be embedded in a computer program product. The computer program product can comprise a computer-readable storage medium in which is embedded a computer program comprising computer-executable code for directing a computing device or computer-based system to perform the various procedures, processes and methods described herein. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed as the best mode contemplated for carrying out this invention, but that the invention will include all embodiments falling within the scope of the appended claims.

The word “comprising”, “comprise”, or “comprises” as used herein should not be viewed as excluding additional elements. The singular article “a” or “an” as used herein should not be viewed as excluding a plurality of elements. The word “or” should be construed as an inclusive or, in other words as “and/or”.

The Abstract of the Disclosure is provided to comply with U.S. rule 37 C.F.R. §1.72(b), requiring an abstract that will allow the reader to quickly ascertain the nature of the technical disclosure. It is submitted with the understanding that it will not be used to interpret or limit the scope or meaning of the claims.

What is claimed is:

1. A method for dynamic metal distortion compensation using an Electromagnetic Tracking System (EMTS) (10) comprising:
generating an electromagnetic field from at least one electromagnetic field generator (12);
providing a plurality of fiducial markers (14), each fiducial marker comprising at least one electromagnetic sensor (26), the electromagnetic sensors oriented in a plurality of sensor orientations, and at least some of the sensors being located proximal to a volume of interest;
imaging the fiducial markers to provide at least a baseline position of the fiducial markers in image space;
monitoring position readings of the plurality of electromagnetic sensors using the EMTS;
calculating a metal distortion correction function by comparing the positions of the fiducial markers in image space to the position readings of the electromagnetic sensors in the electromagnetic field;
monitoring position readings of a medical device (16) moving through the volume of interest using the EMTS, the device having at least one electromagnetic sensor;
applying said distortion correction function to said medical device position readings to compensate for said metal distortion.

2. The method according to claim 1, wherein the positioning of at least one fiducial marker (14) is alterable during the position monitoring.

3. The method according to claim 1, wherein at least some of the fiducial markers (14) are placed on a frame surrounding at least a portion of a patient's body (18) during a medical procedure.

4. The method according to claim 1, wherein at least some of the fiducial markers (14) are placed directly onto a patient's skin during a medical procedure.

5. The method according to claim 1, wherein at least one of the fiducial markers (14) is placed internally in a patient's body (18) during a medical procedure.

6. The method according to claim 1, further comprising selecting some of the position readings of the plurality of electromagnetic sensors (26) to contribute to the metal distortion correction function.

7. The method according to claim 6, wherein the selection of the electromagnetic sensors (26) is dynamically based on selection criteria.

8. The method according to claim 7, wherein the selection criteria comprise selecting sensors (26) with orientations closest to the orientation of the tracked medical device (16) to calculate the compensation.

9. The method according to claim 7, wherein the selection criteria comprise selecting sensors (26) with spatial locations proximal to the spatial location of the tracked medical device (16) to calculate the compensation.

10. The method according to claim 7, wherein the selection criteria comprise selecting sensors (26) with spatial locations proximal to a target location within a patient's body (18) to calculate the compensation.

11. The method according to claim 7, wherein the selection criteria comprise selecting sensors (26) based on the geometry of the relative spatial locations of the tracked medical device (16) and a target location within a patient's body (18) to calculate the compensation.

12. The method according to claim 7, wherein the selection criteria change as at least one of the orientation and spatial location of the tracked medical device changes.

13. The method according to claim 6, wherein the method for calculating the metal distortion correction function is selected based upon the selection of electromagnetic sensors (26).

14. The method according to claim 13, wherein a global transformation calculation method is used.

15. The method according to claim 13, wherein an interpolation calculation is used.

16. The method according to claim 13, wherein a global transformation calculation method is used if the tracked medical device (16) lies outside a geometric coverage of the selected sensors (26).

17. The method according to claim 13, wherein the method for calculating the metal distortion correction function is dynamically changed as the selection of electromagnetic sensors (26) is changed due to movement of the tracked medical device (16).

18. A system for dynamic metal distortion compensation using an Electromagnetic Tracking System (EMTS) (10) comprising:
at least one electromagnetic field generator (12) for generating an electromagnetic field;
a plurality of fiducial markers (14), each fiducial marker comprising at least one electromagnetic sensor (26), the electromagnetic sensors oriented in a plurality of sensor orientations, and at least some of the sensors being located proximal to a volume of interest, the fiducial markers being visible in image space;
a processor for calculating a metal distortion correction function by comparing positions of the fiducial markers in image space to position readings of the electromagnetic sensors in the electromagnetic field; and
at least one electromagnetic sensor attached to a medical device (16),
wherein the processor applies the calculated distortion correction function to said medical device position readings to compensate for said metal distortion.

19. The system according to claim 18, wherein at least some of the fiducial markers (14) are provided on a frame adapted to surround at least a portion of a patient's body (18) during a medical procedure.

20. A computer readable storage medium comprising computer instructions for causing a computing device to:
generate images of fiducial markers (14) to provide at least a baseline position of the fiducial markers in an image space, wherein the fiducial markers comprise electromagnetic sensors (26) oriented in a plurality of sensor orientations, wherein at least some of the sensors are located proximal to a volume of interest, and wherein the sensors can detect an electromagnetic field being generated from at least one electromagnetic field generator (12);
monitor position readings of the electromagnetic sensors;
calculate a metal distortion correction function by comparing the positions of the fiducial markers in the image space to the position readings of the electromagnetic sensors in the electromagnetic field;
monitor position readings of a medical device (16) moving through the volume of interest; and
apply the distortion correction function to the medical device position readings to compensate for the metal distortion.
